(19) United States
(12) Patent Application Publication
(10) Pub. No.: US 2026/0007474 A1
(43) Pub. Date: Jan. 8, 2026

(54) MINIMALLY INVASIVE PUNCTURE SYSTEM BASED ON NAVIGATION SYSTEM

(71) Applicant: SHANGHAI MEDVIDA MEDICAL TECHNOLOGY CO., LTD., Shanghai (CN)

(72) Inventor: Shujun TANG, Shanghai (CN)

(73) Assignee: SHANGHAI MEDVIDA MEDICAL TECHNOLOGY CO., LTD., Shanghai (CN)

(21) Appl. No.: 19/124,868

(22) PCT Filed: Jun. 7, 2023
(86) PCT No.: PCT/CN2023/098970

§ 371 Date: Apr. 27, 2025

(30) Foreign Application Priority Data
May 9, 2023 (CN) 202310516888.3

(51) Int. Cl. A61B 34/20; A61B 10/02; A61B 18/00; A61B 34/00; A61B 90/00

(52) U.S. Cl. CPC A61B 34/20; A61B 10/0233; A61B 18/00; A61B 34/25; A61B 90/37; A61B 2018/00577; A61B 2034/2051; A61B 2090/378

(57) ABSTRACT

The provided is a minimally invasive puncture system based on a navigation system. The minimally invasive puncture system includes a puncture instrument and an electromagnetic navigation module. A first electromagnetic positioning sensor in the electromagnetic navigation module is fixedly provided on a puncture needle body portion of the puncture instrument, and a second electromagnetic positioning sensor is slidably sleeved on the puncture needle body portion. An electromagnetic positioning reading device in the electromagnetic navigation module is configured to acquire real-time coordinates of the first electromagnetic positioning sensor and the second electromagnetic positioning sensor, so as to obtain a real-time movement trajectory of the second electromagnetic positioning sensor relative to the first electromagnetic positioning sensor, thereby determining whether a real-time needle insertion trajectory of the puncture needle body portion entering the human body conforms to a system-default needle insertion trajectory.

### CROSS-REFERENCE TO THE RELATED APPLICATIONS

This application is the national phase entry of International Application No. PCT/CN2023/098970, filed on Jun. 7, 2023, which is based upon and claims priority to Chinese Patent Application No. 202310516888.3, filed on May 9, 2023, the entire contents of which are incorporated herein by reference.

### TECHNICAL FIELD

The present invention relates to the technical field of medical devices, in particular to a minimally invasive puncture system based on a navigation system.

### BACKGROUND

Ablation tools such as a puncture tool and a biopsy tool are applied in surgery with the characteristics of small trauma, easy grasp in surgery, short time during surgery, etc. An ablation device has less trauma during an ablation surgery, and thus has been widely used in clinical practice for the thermal coagulation of tumor tissues of liver, lung, kidney and other organs. With the development of technologies, ablation itself has also developed in a direction of becoming more and more precise and controllable. However, due to the limitations of the technologies themselves, it is impossible to intuitively determine whether a relative position of the ablation device to a lesion is accurate, so impossible to accurately determine and monitor an ablation range during use, and further impossible to accurately place a puncture or biopsy tool in a target sampling area, thereby affecting the sampling efficiency of biopsies.

### SUMMARY

In view of the above-mentioned defects in the prior art, the present invention provides a minimally invasive puncture system based on a navigation system, which effectively assists operators in intuitively determining whether relative positions of a puncture device and a lesion effectively coincide, with easy operation, improved ablation and high operation precision.

To fulfill said objective, the minimally invasive puncture system based on the navigation system has the following composition:
the minimally invasive puncture system based on the navigation system is mainly characterized by including:
a puncture instrument, the puncture instrument including a handheld portion and a puncture needle body portion, the puncture needle body portion being disposed on the handheld portion;
an electromagnetic navigation module, the electromagnetic navigation module including an electromagnetic positioning reading device, a first electromagnetic positioning sensor and a second electromagnetic positioning sensor, wherein the first electromagnetic positioning sensor is fixedly provided on a position of the puncture needle body portion adjacent to the handheld portion, the second electromagnetic positioning sensor is slidably sleeved on the puncture needle body portion, and the second electromagnetic positioning sensor is located on one side of the first electromagnetic positioning sensor away from the handheld portion; and
the electromagnetic positioning reading device is configured to acquire real-time coordinates of the first electromagnetic positioning sensor and the second electromagnetic positioning sensor, so as to obtain a real-time movement trajectory of the second electromagnetic positioning sensor relative to the first electromagnetic positioning sensor, thereby determining whether a real-time needle insertion trajectory of the puncture needle body portion entering the human body conforms to a system-default needle insertion trajectory.

According to the above minimally invasive puncture system based on the navigation system, the minimally invasive puncture system based on the navigation system further includes:
an ultrasound imaging module, the ultrasound imaging module including an ultrasound probe, and the ultrasound imaging module being configured to generate a real-time ultrasound image based on detection information from the ultrasound probe;
the electromagnetic navigation module further including a third electromagnetic positioning sensor, the third electromagnetic positioning sensor being provided on the ultrasound probe;
the electromagnetic positioning reading device being further configured to acquire real-time coordinates of the third electromagnetic positioning sensor, so as to determine a relative position relationship between the ultrasound probe and the puncture needle body portion according to the real-time coordinates of the first electromagnetic positioning sensor, the second electromagnetic positioning sensor and the third electromagnetic positioning sensor;
an image fusion module, configured to fuse the real-time needle insertion trajectory of the puncture needle body portion entering the human body with the real-time ultrasound image, and generate a device navigation screen in which a relative spatial position of the puncture needle body portion is fused into the real-time ultrasound image; and
a display module, configured to receive the device navigation screen generated by the image fusion module and display the device navigation screen.

According to the minimally invasive puncture system based on the navigation system, the system-default needle insertion trajectory of the puncture needle body portion entering the human body is also displayed in the device navigation screen.

According to the minimally invasive puncture system based on the navigation system, the image fusion module includes:
an ultrasound image calibration unit, configured to construct a relative position and angle relationship between the real-time ultrasound image and the third electromagnetic positioning sensor, and determine a mapping relationship between each pixel point in the real-time ultrasound image and the third electromagnetic positioning sensor;
an ablation probe calibration unit, configured to construct a mapping relationship between the real-time coordinates of the first electromagnetic positioning sensor and the second electromagnetic positioning sensor and the real-time needle insertion trajectory of the puncture needle body portion; and
a coordinate unification unit, configured to transform mean-value coordinates of each pixel point in the real-time ultrasound image and the real-time needle insertion trajectory of the puncture needle body portion into a reference coordinate system formed by a magnetic field generator in the electromagnetic navigation module to generate the device navigation screen.

According to the minimally invasive puncture system based on the navigation system, the image fusion module further includes:
a three-dimensional reconstruction unit, configured to transform a two-dimensional image acquired by the ultrasound imaging module into a three-dimensional ultrasound image.

According to the minimally invasive puncture system based on the navigation system, in the event of fusing the real-time needle insertion trajectory of the puncture needle body portion entering the human body with the real-time ultrasound image, and generating the device navigation screen in which the relative spatial position of the puncture needle body portion is fused into the real-time ultrasound image, the following operations are performed:
obtaining a real-time movement trajectory of the second electromagnetic positioning sensor relative to the first electromagnetic positioning sensor according to the acquired real-time coordinates of the first electromagnetic positioning sensor and the second electromagnetic positioning sensor, so as to acquire the real-time needle insertion trajectory of the puncture needle body portion entering the human body;
determining a mapping relationship between the reference coordinate system formed by the magnetic field generator in the electromagnetic navigation module and the real-time ultrasound image;
determining a mapping relationship between the real-time needle insertion trajectory of the puncture needle body portion entering the human body and the reference coordinate system; and
generating the device navigation screen in which the relative spatial position of the puncture needle body portion is fused into the real-time ultrasound image according to the mapping relationship between the reference coordinate system and the real-time ultrasound image and the mapping relationship between the real-time needle insertion trajectory of the puncture needle body portion entering the human body and the reference coordinate system.

According to the minimally invasive puncture system based on the navigation system, the determining the mapping relationship between the reference coordinate system formed by the magnetic field generator in the electromagnetic navigation module and the real-time ultrasound image includes:
performing coordinate system relationship transformation and unification on an ultrasound probe coordinate system where the third electromagnetic positioning sensor is located relative to the reference coordinate system, so as to determine the mapping relationship between the reference coordinate system and the real-time ultrasound image.

According to the minimally invasive puncture system based on the navigation system, the determining the mapping relationship between the real-time needle insertion trajectory of the puncture needle body portion entering the human body and the reference coordinate system includes:
performing coordinate system relationship transformation and unification on a puncture needle body coordinate system where the first electromagnetic positioning sensor and the second electromagnetic positioning sensor are located relative to the reference coordinate system, so as to determine the mapping relationship between the real-time needle insertion trajectory of the puncture needle body portion entering the human body and the reference coordinate system.

According to the minimally invasive puncture system based on the navigation system, the generating the device navigation screen in which the relative spatial position of the puncture needle body portion is fused into the real-time ultrasound image according to the mapping relationship between the reference coordinate system and the real-time ultrasound image and the mapping relationship between the real-time needle insertion trajectory of the puncture needle body portion entering the human body and the reference coordinate system includes:
determining a relative position relationship between the real-time ultrasound image in the reference coordinate system and the real-time needle insertion trajectory of the puncture needle body portion entering the human body according to the mapping relationship between the reference coordinate system and the real-time ultrasound image and the mapping relationship between the real-time needle insertion trajectory of the puncture needle body portion entering the human body and the reference coordinate system, and further generating the device navigation screen in which the relative spatial position of the puncture needle body portion is fused into the real-time ultrasound image.

According to the minimally invasive puncture system based on the navigation system, the puncture instrument may be any one of a puncture set, an ablation device and a biopsy tool.

The minimally invasive puncture system based on the navigation system of the present invention has the following beneficial effects.

1. Compared with a puncture device without a navigation positioning sensor, the minimally invasive puncture system based on the navigation system of the present invention is used such that a spatial position of a tip of the puncture needle body portion may be inferred by using real-time positions of the first electromagnetic positioning sensor and the second electromagnetic positioning sensor, and then tracked and displayed, so the operator can adjust a puncture direction of the puncture instrument in real time according to a spatial position of the real-time needle insertion trajectory in use.

2. Two or more first electromagnetic positioning sensors are provided on the puncture needle body portion in the minimally invasive puncture system based on the navigation system, wherein one of the electromagnetic positioning sensors provided at the end of the puncture needle body portion is designed in a fixed mode, and the coordinates of the fixed electromagnetic positioning sensor are used as basic coordinates of the puncture needle body coordinate system. Other electromagnetic positioning sensors are designed to be slidably structured. During the puncture process, the slidable electromagnetic positioning sensor can move toward the fixed electromagnetic positioning sensor under the push of skin. The electromagnetic navigation module may determine a relative position of the movable electromagnetic positioning sensor on the puncture needle body portion according to a change in a relative position of the fixed electromagnetic positioning sensor on the movable electromagnetic positioning sensor, and determine whether the puncture needle body portion has bent and deformed according to relative movement trajectories of the plurality of electromagnetic positioning sensors on the puncture needle body portion. Because the electromagnetic positioning sensor near the tip of the puncture needle body portion in this scheme is slidably designed, the thickness of the needle body entering the human body cannot increase, and at the same time, for some puncture instruments that need to work at high temperature, the high temperature at the tip will not affect the electromagnetic positioning sensor. This method not only gives designers more convenience, but also improves the precision of the system.

3. The spatial position of the needle insertion trajectory of the puncture needle body portion is positioned and displayed through the display module, and the spatial position of the tip of the puncture needle body portion can be acquired before the puncture needle body portion is inserted into a solid organ. In the actual use process, the spatial position can assist the puncture needle body portion in planning a needle entry point position where the puncture needle body portion and the surface of an organ (e.g., liver) are crossed.

4. The reconstructed ultrasound image is displayed on the display in real time, and in a real-time displayed image, the position of the puncture needle body portion is dynamically displayed in a display screen, which facilitates real-time adjustment of the puncture needle body portion during actual use through the displayed dynamic image. It is ensured that an electrode needle is placed accurately on the target ablation lesion area.

### BRIEF DESCRIPTION OF THE DRAWINGS

The conception, specific structures and produced technical effects of the present invention will be further described in conjunction with the accompanying drawings, so as to fully understand the purpose, characteristics and effects of the present invention.

FIG. 1 is a module relationship diagram of a minimally invasive puncture system based on a navigation system in one embodiment of the present invention.

FIG. 2 is a relative position relationship diagram of an ablation probe, an ablation handle, a first electromagnetic positioning sensor and a second electromagnetic positioning sensor in one embodiment of the present invention.

FIG. 3 is a relative position diagram of a second electromagnetic positioning sensor in a first state on an ablation probe in one embodiment of the present invention.

FIG. 4 is a relative position diagram of a second electromagnetic positioning sensor in a second state on an ablation probe in one embodiment of the present invention.

FIG. 5 is a schematic diagram of a needle entry state with human tissues.

FIG. 6A is a schematic diagram of a device navigation screen of a minimally invasive puncture system based on a navigation system in one embodiment of the present invention.

FIG. 6B is a schematic diagram of a device navigation screen of a minimally invasive puncture system based on a navigation system in another embodiment of the present invention.

FIG. 7 is a state schematic diagram of a puncture needle body portion.

FIGS. 8A-8C show a scan-mapping relationship based on an N-line calibration block.

Reference symbols represent the following components:
1 handheld portion
2 puncture needle body portion
3 first electromagnetic positioning sensor
4 second electromagnetic positioning sensor
5 real-time needle insertion trajectory
6 system-default needle insertion trajectory
7 human epidermal tissue area
8 lesion
9 needle entry point

### DETAILED DESCRIPTION OF THE EMBODIMENTS

In order to make the technical means, creative features, objectives and effects achieved in the present invention be easy to understand, the present invention is further elaborated in combination with specific diagrams. However, the present invention is not limited to the following embodiments.

It should be noted that the structure, ratio, size, etc. shown in the accompanying drawings in the present specification are only used to match the content disclosed in the specification for those skilled in the art to understand and read, are not regarded as limiting conditions for the implementation of the present invention, and therefore have no technical significance. Any structural modification, proportional relationship change or size adjustment should still fall within the scope of the technical content disclosed in the present invention, without affecting the effects and objectives that can be achieved by the present invention.

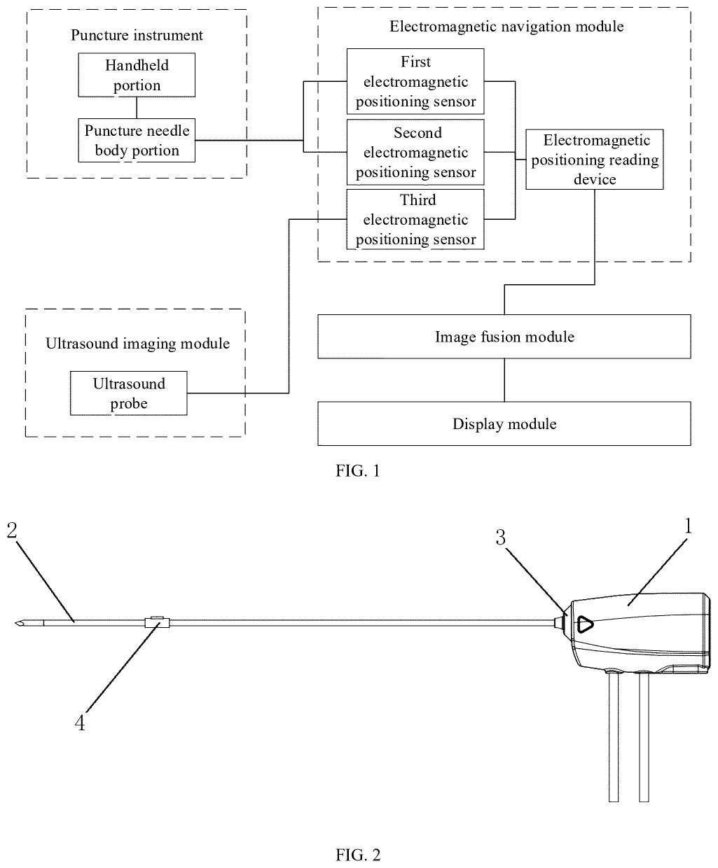

The present invention is further described below in conjunction with FIGS. 1-5, FIGS. 6A-6B, FIG. 7, and FIGS. 8A-8C, wherein FIG. 1 only reflects an association relationship among various modules and components therein, without limiting an actual design position of the components.

A minimally invasive puncture system based on a navigation system in this embodiment includes:
a puncture instrument, the puncture instrument including a handheld portion 1 and a puncture needle body portion 2, the puncture needle body portion 2 being provided on the handheld portion 1; and
an ultrasound imaging module, the ultrasound imaging module including an ultrasound probe, and the ultrasound imaging module being configured to generate a real-time ultrasound image based on detection information from the ultrasound probe;
an electromagnetic navigation module, the electromagnetic navigation module including an electromagnetic positioning reading device, a first electromagnetic positioning sensor 3, a second electromagnetic positioning sensor 4 and a third electromagnetic positioning sensor, wherein as shown in FIG. 2, the first electromagnetic positioning sensor 3 is fixedly provided on a position of the puncture needle body portion 2 adjacent to the handheld portion 1, the second electromagnetic positioning sensor 4 is slidably sleeved on the puncture needle body portion 2, and the second electromagnetic positioning sensor 4 is located on one side of the first electromagnetic positioning sensor 3 away from the handheld portion 1; the third electromagnetic positioning sensor is provided on the ultrasound probe;
the electromagnetic positioning reading device is configured to acquire real-time coordinates of the first electromagnetic positioning sensor 3 and the second electromagnetic positioning sensor 4, so as to obtain a real-time movement trajectory of the second electromagnetic positioning sensor 4 relative to the first electromagnetic positioning sensor 3, thereby determining whether a real-time needle insertion trajectory 5 of the puncture needle body portion 2 entering the human body conforms to a system-default needle insertion trajectory 6;
the electromagnetic positioning reading device is further configured to acquire real-time coordinates of the third electromagnetic positioning sensor, so as to determine a relative position relationship between the ultrasound probe and the puncture needle body portion 2 according to the real-time coordinates of the first electromagnetic positioning sensor 3, the second electromagnetic positioning sensor 4 and the third electromagnetic positioning sensor;
an image fusion module, configured to fuse the real-time needle insertion trajectory 5 of the puncture needle body portion 2 entering the human body with the real-time ultrasound image, and generate a device navigation screen in which a relative spatial position of the puncture needle body portion 2 is fused into the real-time ultrasound image; and
a display module, configured to receive the device navigation screen generated by the image fusion module and display the device navigation screen.

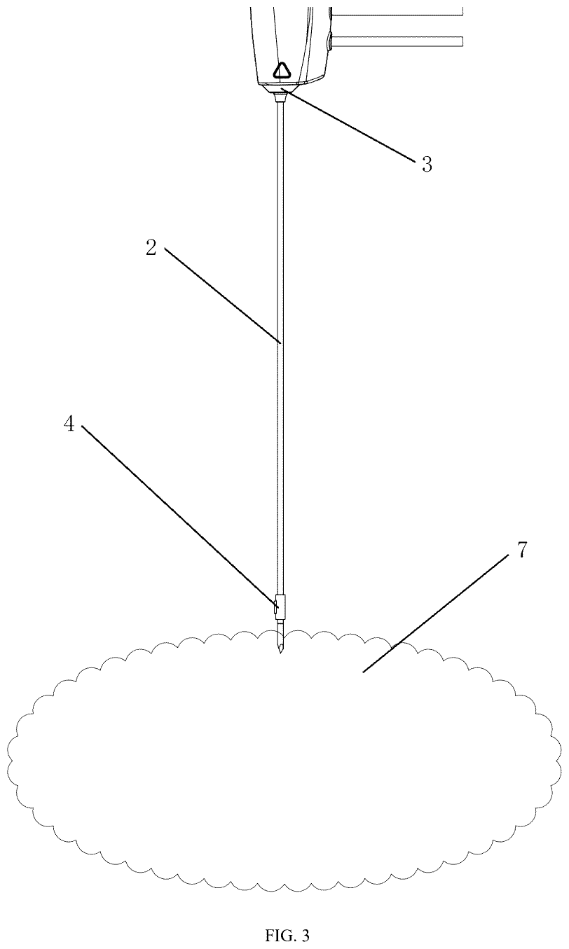

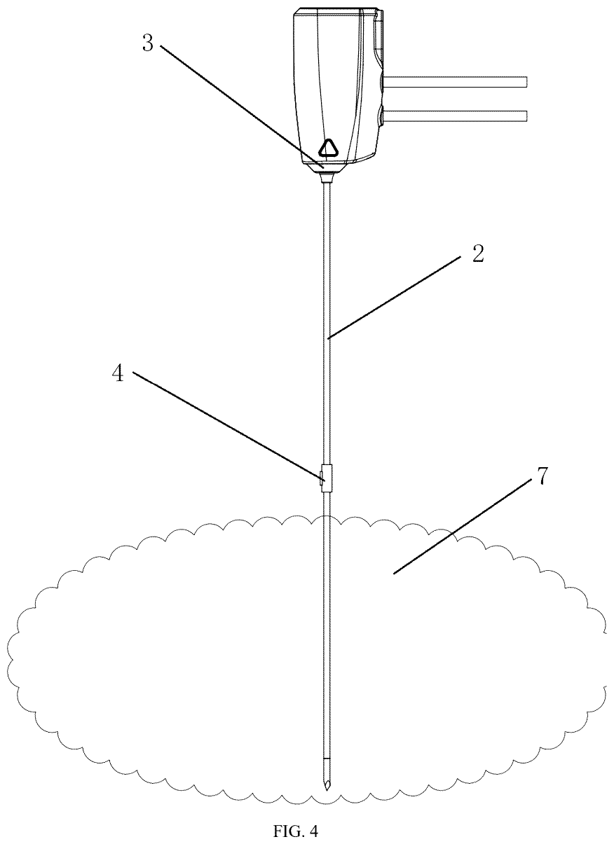

As shown in FIG. 3 and FIG. 4, the second electromagnetic positioning sensor 4 may slide on the puncture needle body portion 2. During the actual surgical operation, the second electromagnetic positioning sensor 4 may be first provided at a position shown in FIG. 3, that is, a position close to a tip of the puncture needle body portion 2. Then, as the puncture needle body portion 2 gradually enters the human body, the second electromagnetic positioning sensor 4 can slowly approach to the first electromagnetic positioning sensor 3 under the push of a human epidermal tissue area 7, that is, in a state shown in FIG. 4, and the electromagnetic navigation module captures relative positions of the first electromagnetic positioning sensor 3 and the second electromagnetic positioning sensor 4.

The electromagnetic navigation module may determine whether the second electromagnetic positioning sensor 4 moves toward the first electromagnetic positioning sensor 3 along a preset trajectory (i.e., a trajectory formed by the puncture needle body portion 2 in a normal state is prestored in the system) by detecting coordinates of the first electromagnetic positioning sensor 3 and the second electromagnetic positioning sensor 4. If some puncture needle body portions 2 are deformed under the action of an external force in the puncture process, the second electromagnetic positioning sensor 4 cannot move toward the first electromagnetic positioning sensor 3 along the preset trajectory at this moment. Then, the real-time needle insertion trajectory 5 of the puncture needle body portion 2 entering the human body will not conform to the system-default needle insertion trajectory 6. At this time, the puncture abnormality can be found (FIG. 7 is a state schematic diagram of the puncture needle body portion 2, where a solid line portion is a schematic diagram of the puncture needle body portion 2 in a normal state, while a dotted line portion is an outline diagram of the puncture needle body portion 2 under the condition of bending). Alternatively, because an operation mode of the operator is incorrect, the puncture needle body portion 2 cannot be punctured to a lesion 8. At this time, the real-time needle insertion trajectory 5 of the puncture needle body portion 2 entering the human body can still be obtained according to the coordinates of the first electromagnetic positioning sensor 3 and the second electromagnetic positioning sensor 4, and the tip position of the puncture needle body portion 2 cannot be made to reach the lesion 8 according to the current operation mode.

In the present invention, a spatial position of the tip of the puncture needle body portion 2 may be inferred by using acquired real-time positions of the first electromagnetic positioning sensor 3 and the second electromagnetic positioning sensor 4, and then tracked and displayed, so an operator can adjust a puncture direction of the puncture instrument in real time according to a spatial position of the real-time needle insertion trajectory 5 in use, thereby effectively assisting the operator in performing accurate operations.

In the actual operation process, a method of using a screen for real-time display in this embodiment may be used for prompt, or a method of voice early warning may also be used for early warning, but a method of voice early warning is less intuitive than the method of displaying the real-time needle insertion trajectory 5 by using the screen.

In this embodiment, a relative spatial position of the puncture needle body portion 2 is also fused with a real-time ultrasound image, so that the operator can determine a relative position relationship between the needle insertion trajectory and a point location of the lesion 8 more intuitively.

In this embodiment, the system-default needle insertion trajectory 6 of the puncture needle body portion 2 entering the human body is also displayed in the device navigation screen.

Because the system-default needle insertion trajectory 6 is also displayed in the navigation screen, the operator can be well guided to carry out a needle insertion operation.

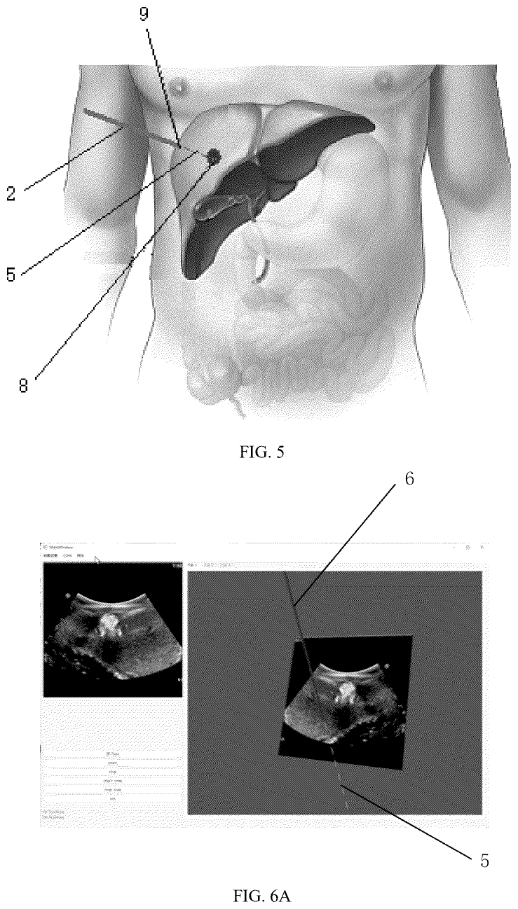

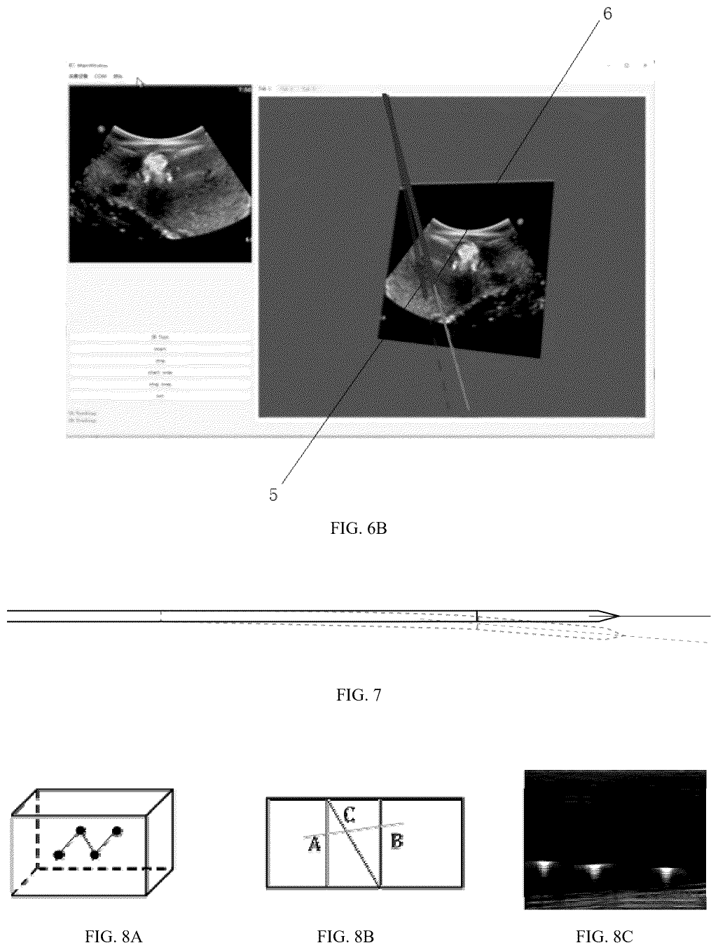

It should be noted that in the practical operation process, the real-time needle insertion trajectory 5 is a guide line that can predict an advance direction of the needle body, rather than a line that coincides with the needle body. FIG. 5 is a schematic diagram of a needle entry state with human tissues. As shown in FIG. 5, the lesion 8 is located at the liver position of the human body, and the puncture needle body portion 2 (due to a picture size relationship, only a thicker solid line is drawn in FIG. 5 to represent the puncture needle body portion 2, without limiting an actual shape of the puncture needle body portion 2) is currently at a needle entry point 9 outside the human body. At this time, the real-time needle insertion trajectory 5 is formed by a dotted line extending from the needle entry point 9 in FIG. 5 to the lesion 8. At this time, a screen that the operator sees in the display module may be shown in FIG. 6A or FIG. 6B.

FIG. 6A and FIG. 6B are schematic diagrams of two device navigation screens of a minimally invasive puncture system based on a navigation system, respectively. In FIG. 6A, a solid line is the system-default needle insertion trajectory 6, and a dotted line is the real-time needle insertion trajectory 5. It can be seen from FIG. 6A that the real-time needle insertion trajectory 5 coincides with the system-default needle insertion trajectory 6, a needle insertion direction can be then determined at this moment accurately, and the operator can perform the needle insertion operation normally. However, in FIG. 6B, a line on the left side is the real-time needle insertion trajectory 5 (a solid line portion in the real-time needle insertion trajectory 5 represents a position where the puncture needle body portion 2 is located, and a dotted line portion indicates a position that the puncture needle body portion 2 will reach if it continues to advance according to the current trajectory; a line on the right side is the system-default needle insertion trajectory 6; and the operator can adjust a needle insertion direction according to deviations of the real-time needle insertion trajectory 5 and the system-default needle insertion trajectory 6, so as to perform a calibration operation.

The real-time needle insertion trajectory 5 in the figure is a predicted needle entry trajectory formed according to the coordinates of the current first electromagnetic positioning sensor 3 and the second electromagnetic positioning sensor 4 and a relative position change relationship therebetween. That is, a needle insertion direction and position present by the operator during operation according to the current operation direction are drawn for the operator to determine whether to continue to carry out the needle insertion operation according to the current needle insertion trajectory, or to make adaptive adjustments.

The image fusion module includes:
an ultrasound image calibration unit, configured to construct a relative position and angle relationship between the real-time ultrasound image and the third electromagnetic positioning sensor, and determine a mapping relationship between each pixel point in the real-time ultrasound image and the third electromagnetic positioning sensor;
an ablation probe calibration unit, configured to construct a mapping relationship between the real-time coordinates of the first electromagnetic positioning sensor 3 and the second electromagnetic positioning sensor 4 and the real-time needle insertion trajectory 5 of the puncture needle body portion 2; and
a coordinate unification unit, configured to transform mean-value coordinates of each pixel point in the real-time ultrasound image and the real-time needle insertion trajectory 5 of the puncture needle body portion 2 into a reference coordinate system formed by a magnetic field generator in the electromagnetic navigation module to generate the device navigation screen; and
a three-dimensional reconstruction unit, configured to transform a two-dimensional image acquired by the ultrasound imaging module into a three-dimensional ultrasound image.

In the event of fusing the real-time needle insertion trajectory 5 of the puncture needle body portion 2 entering the human body with the real-time ultrasound image and generating the device navigation screen in which the relative spatial position of the puncture needle body portion 2 is fused into the real-time ultrasound image, the following operations are performed:
obtaining a real-time movement trajectory of the second electromagnetic positioning sensor 4 relative to the first electromagnetic positioning sensor 3 according to the acquired real-time coordinates of the first electromagnetic positioning sensor 3 and the second electromagnetic positioning sensor 4, so as to acquire the real-time needle insertion trajectory 5 of the puncture needle body portion 2 entering the human body;
determining a mapping relationship between the reference coordinate system formed by the magnetic field generator in the electromagnetic navigation module and the real-time ultrasound image, which specifically includes:
performing coordinate system relationship transformation and unification on an ultrasound probe coordinate system where the third electromagnetic positioning sensor is located relative to the reference coordinate system, so as to determine a mapping relationship between the reference coordinate system and the real-time ultrasound image (in the implementation process, the relative position of each pixel point in the ultrasound image relative to a coordinate point of the third electromagnetic positioning sensor can be fixed, and then a mapping relationship between each pixel point in the ultrasound image and the reference coordinate system can be determined after a relationship between the coordinate point of the third electromagnetic positioning sensor and the reference coordinate system is determined);
determining a mapping relationship between the real-time needle insertion trajectory 5 of the puncture needle body portion 2 entering the human body and the reference coordinate system, which specifically includes:
performing coordinate system relationship transformation and unification on a puncture needle body coordinate system where the first electromagnetic positioning sensor 3 and the second electromagnetic positioning sensor 4 are located relative to the reference coordinate system, so as to determine the mapping relationship between the real-time needle insertion trajectory 5 of the puncture needle body portion 2 entering the human body and the reference coordinate system (in the implementation process, the coordinates of the first electromagnetic positioning sensor 3 and the second electromagnetic positioning sensor 4 may correspond to a contour relationship of the puncture needle body portion 2, so that the real-time needle insertion trajectory 5 of the puncture needle body portion 2 entering the human body may be determined according to the coordinates of the first electromagnetic positioning sensor 3 and the second electromagnetic positioning sensor 4 and the movement trajectory of the second electromagnetic positioning sensor 4 relative to the first electromagnetic positioning sensor 3, so the mapping relationship between the real-time needle injection trajectory 5 and the reference coordinate system may be determined after the coordinates of the first electromagnetic positioning sensor 3 and the second electromagnetic positioning sensor 4 are mapped to the reference coordinate system); and
generating the device navigation screen in which the relative spatial position of the puncture needle body portion 2 is fused into the real-time ultrasound image according to the mapping relationship between the reference coordinate system and the real-time ultrasound image and the mapping relationship between the real-time needle insertion trajectory 5 of the puncture needle body portion 2 entering the human body and the reference coordinate system, which specifically includes:
   determining a relative position relationship between the real-time ultrasound image in the reference coordinate system and the real-time needle insertion trajectory 5 of the puncture needle body portion 2 entering the human body according to the mapping relationship between the reference coordinate system and the real-time ultrasound image and the mapping relationship between the real-time needle insertion trajectory 5 of the puncture needle body portion 2 entering the human body and the reference coordinate system, and further generating the device navigation screen in which the relative spatial position of the puncture needle body portion 2 is fused into the real-time ultrasound image.

That is, in the process of performing the generation process of the device navigation screen, the electromagnetic navigation module respectively performs coordinate system relationship transformation and unification on a puncture needle body coordinate system where the first electromagnetic positioning sensor 3 and the second electromagnetic positioning sensor 4 are located and an ultrasound probe coordinate system where the third electromagnetic positioning sensor is located relative to the reference coordinate system formed by the magnetic field generator in the electromagnetic navigation module, such that after the coordinates of the first electromagnetic positioning sensor 3, the second electromagnetic positioning sensor 4 and the third electromagnetic positioning sensor are unified into the same coordinate system, a relative coordinate relationship among the first electromagnetic positioning sensor 3, the second electromagnetic positioning sensor 4 and the third electromagnetic positioning sensor is determined in this unified coordinate system; a relative position relationship between the ultrasound probe and the puncture needle body portion 2 is further determined; and the real-time needle insertion trajectory 5 of the puncture needle body portion 2 entering the human body is presented in the same screen with the real-time ultrasound image.

In the mapping process of various coordinates, in the puncture needle body coordinate system, the coordinates (x1,y1,z1) of the first electromagnetic positioning sensor 3 may be used as reference coordinates of the puncture needle body coordinate system; and in the ultrasound probe coordinate system, the coordinates (x2,y2,z2) of the third electromagnetic positioning sensor may be used as reference coordinates of the ultrasound probe coordinate system. The puncture needle body coordinate system and the ultrasound probe coordinate system are subjected to coordinate relationship transformation and unification relative to the reference coordinate system formed by the magnetic field generator in the electromagnetic navigation module, and a relative position relationship between the coordinates (x1, y1, z1) of the first electromagnetic positioning sensor 3 and the coordinates (x2, y2, z2) of the third electromagnetic positioning sensor and reference coordinates (x, y, z) in the reference coordinate system is determined. Further, through a transformation relationship of the above coordinate systems, the relative positions among various coordinate systems can be clearly understood (the specific coordinate transformation mode adopted in this embodiment may be performed by adopting a coordinate unified transformation method in the prior art). In this embodiment, because the third electromagnetic positioning sensor is provided on the ultrasound probe, point location space coordinates on the ultrasound image can be acquired by utilizing the third electromagnetic positioning sensor. Therefore, the ultrasound image may be displayed in the electromagnetic positioning sensor, and then a relative position relationship among the puncture needle body portion 2, the real-time needle insertion trajectory 5 and the real-time ultrasound image is determined in the same screen.

In the implementation process, two or more first electromagnetic positioning sensors 3 are provided on the puncture needle body portion 2, wherein one of the electromagnetic positioning sensors provided at the end of the puncture needle body portion 2 is designed in a fixed mode, and the coordinates of the fixed electromagnetic positioning sensor are used as basic coordinates of the puncture needle body coordinate system. Other electromagnetic positioning sensors are designed to be slidably structured. During the puncture process, the slidable electromagnetic positioning sensor can move toward the fixed electromagnetic positioning sensor under the push of skin. The electromagnetic navigation module may determine a relative position of the movable electromagnetic positioning sensor on the puncture needle body portion 2 according to a change in a relative position of the fixed electromagnetic positioning sensor to the movable electromagnetic positioning sensor, and determine whether the puncture needle body portion 2 has bent and deformed according to relative movement trajectories of the plurality of electromagnetic positioning sensors on the puncture needle body portion 2. Because the electromagnetic positioning sensor near the tip of the puncture needle body portion 2 in this scheme is slidably designed, the thickness of the needle body entering the human body cannot increase, and at the same time, for some puncture instruments that need to work at high temperature, the high temperature at the tip will not affect the electromagnetic positioning sensor. This method not only gives designers more convenience, but also improves the precision of the system.

The puncture instrument in the above embodiment may be any one of a puncture set, an ablation device and a biopsy tool.

When the puncture instrument is the ablation device, the puncture instrument further includes an ablation host, and an ablation handle constitutes the handheld portion 1; and an ablation probe constitutes the puncture needle body portion 2.

The ablation handle is connected to the ablation host.

The ablation host interacts with the electromagnetic navigation module, the ultrasound imaging module and the image fusion module.

During application, a position and attitude of the ablation probe are determined by detecting the relative positions of the first electromagnetic positioning sensor 3 and the second electromagnetic positioning sensor 4 on the ablation probe, the real-time needle insertion trajectory 5 is further generated, and the real-time needle insertion trajectory 5 of the ablation probe is fused with the real-time ultrasound image and displayed on the device navigation screen. The minimally invasive puncture system based on the navigation system can effectively assist the operator in intuitively observing relative positions of the operated instrument and the lesion, is easy to operate, and effectively improves the puncture and placement precision of the instrument and the accuracy of biopsy or ablation.

The system combines an electromagnetic navigation technology with ultrasound pattern reconstruction. Through the ultrasound pattern construction, a spatial position of an electrode of the ablation probe and a target tumor is displayed in real time in the constructed ultrasound reconstruction image. The real-time display facilitates the operator to intuitively understand relative spatial positions of the ablation electrode and a target surgical organ as well as adjacent organs, so as to effectively improve the surgical precision and accurately place the ablation electrode in a target area to be ablated under the guidance of navigation (the corresponding technical effect can also be achieved when this technical solution is applied to puncture, biopsy and other schemes). Meanwhile, since the system is provided with two electromagnetic positioning sensors, more design space is reserved in the product implementation.

In the design and process implementation in the prior art, the sensor can usually only be placed at a position close to the handle, rather than at a tip of the ablation probe. However, during the use of an electrode needle, a deformation of the electrode needle possibly caused during the puncture process will lead to artificial errors. If an error beyond an allowable range cannot be found in time, a large deviation of puncture will be caused.

Therefore, in order to solve the above problem, it is designed in the present invention that at least two electromagnetic positioning sensors are provided on the electrode needle (e.g., in some embodiments, three electromagnetic positioning sensors may also be provided on the electrode needle), and the increased slidable electromagnetic positioning sensor may be placed close to the tip of the electrode needle and may be moved during use. At the beginning of use, the electromagnetic positioning sensor may be placed close to the tip at the beginning of the puncture. By reading this position, the system may calculate a position of the tip (the calculated position of the tip is more accurate as it is closer to the tip). Meanwhile, through a relative position to the sensor close to the handle, it may be calculated whether the electrode needle is bent during use, the corresponding real-time needle insertion trajectory 5 is generated, and a user (a display method for the real-time needle insertion trajectory 5 may be shown in FIG. 6A and FIG. 6B) is reminded to adjust the electrode needle to a relatively straight state before puncture. In this way, the increased slidable electromagnetic positioning sensor allows the system to balance ease of use and navigation accuracy.

The core in the navigation system is to acquire corresponding spatial information of the ultrasound image and the ablation/biopsy tool, thereby providing surgical guidance for doctors in a system software interface. During implementation, based on the electromagnetic navigation module, it is necessary to determine a mapping relationship between the ultrasound image and the electromagnetic navigation system, and a mapping relationship between the position and attitude of the ablation tool (puncture or biopsy tool) and the electromagnetic navigation system. In the present invention, a corresponding coordinate system transformation matrix is acquired through a calibration process.

An image processing principle in the above embodiment is further described in conjunction with FIGS. 8A-8C below.

In this embodiment, the image fusion module includes:
an ultrasound image calibration unit, configured to construct a relative position and angle relationship between the real-time ultrasound image and the electromagnetic positioning sensor, and determine a mapping relationship between each pixel point in the real-time ultrasound image and the electromagnetic positioning sensor.

As shown in FIGS. 8A-8C (FIG. 8A represents an N-line calibration block, FIG. 8B represents a scanning diagram, and FIG. 8C represents an actual scanning effect), during specific implementation, by using the calibration block, such as a common N-line calibration block, an actual physical position of an intermediate point C is determined from the positions of points A and Bon FIG. 8B according to a geometric relationship of similar triangles, so as to establish the following equation 1 as a constraint equation:

$${x}_{c}^{w} = [ \begin{matrix}{x}_{c}^{w} \\ {y}_{c}^{w} \\ {z}_{c}^{w} \\ 1\end{matrix} ] = {T}_{g \to w} \cdot {T}_{s \to g} \cdot {T}_{i \to s} {x}_{c}^{i} ; \qquad equation 1$$

wherein T_{s→g} represents a transformation matrix from the coordinate system of each electromagnetic positioning sensor to a magnetic field generator coordinate system (i.e., the reference coordinate system), and T_{g→w} represents a transformation matrix from the magnetic field generator coordinate system to a world coordinate system defined by the N-line calibration block.

$${x}_{c}^{w} and {x}_{c}^{i}$$

represent nomogeneous coordinate representations of the point C in the image coordinate system and the world coordinate system, respectively. A plurality of constraint equations are established by scanning images at different positions and in different directions. The mapping relationship between each pixel point on the ultrasound image and the third electromagnetic positioning sensor is established by applying an iterative optimization algorithm, e.g., solving a transformation matrix T_{i→s}.

An ablation probe calibration unit is configured to construct a relative attitude and offset between the tip position and attitude (i.e., the real-time needle insertion trajectory 5) of the ablation probe and the two electromagnetic positioning sensors.

During implementation, the position and attitude of each electromagnetic positioning sensor in the magnetic field generator coordinate system are known, the puncture needle body portion 2 is placed in parallel with each electromagnetic positioning sensor, and the attitude of each electromagnetic positioning sensor may be kept consistent with the puncture needle body portion 2. A translation amount is obtained by calibration. The calibration method is as follows: a translation matrix from a coordinate system (tip) of the puncture needle body portion 2 to the electromagnetic positioning sensor is denoted as T_{t→s}, and homogeneous coordinates of a point in space in the two coordinate systems are x^{t}=[x^{t}, y^{t}, z^{t}, 1]^{T} and x^{s}=[x^{s}, y^{s}, z^{s}, 1]^{T}, then x^{s}=T_{t→s}x^{t}. A translation matrix T_{t→s} is calculated by using a calibration tool of an electromagnetic positioning system.

Two or more electromagnetic positioning sensors are placed on the puncture needle body portion 2 to be used for precise positioning, which is convenient for the operator to operate and use. For ease of explanation, a working mode of the two electromagnetic positioning sensors is taken as an example below. A spatial transformation relationship in an algorithm is as follows:
the first electromagnetic positioning sensor 3 is a fixed sensor, and a position transformation relationship between the tip position and the first electromagnetic positioning sensor 3 is determined by the mapping relationship mentioned above. The second electromagnetic positioning sensor 4 is a micro-sliding sensor, and a moving direction of the second electromagnetic positioning sensor 4 is used as the basis for determining whether the tip of the puncture needle body portion 2 is bent. The direction vectors of the first electromagnetic positioning sensor 3 and the second electromagnetic positioning sensor 4 are \vec{d₁} and \vec{d₂}. Then, if

$$\vert \frac{\vec{{d}_{1}} \cdot \vec{{d}_{2}}}{‖ \vec{{d}_{1}} ‖ \times ‖ \vec{{d}_{2}} ‖} - 1 \vert < ε$$

is satisfied, it means that the tip does not produce a curvature beyond a preset standard, where a value of ε is 0.01; and if

$$\vert \frac{\vec{{d}_{1}} \cdot \vec{{d}_{2}}}{‖ \vec{{d}_{1}} ‖ \times ‖ \vec{{d}_{2}} ‖} - 1 \vert < ε$$

is not satisfied, it means that the tip produces a curvature beyond the preset standard. The system stops a guidance prompt, and then continues the guidance prompt when the tip returns to the center.

During use, the second electromagnetic positioning sensor 4 is not fixed in position, and after the puncture needle body portion 2 enters the human body, moves toward the handheld portion 1. During use, the first electromagnetic positioning sensor 3 is fixed in position, and placed at a position on the puncture needle body portion 2 adjacent to the handheld portion 1, or may be directly provided inside the handle. During use, the first electromagnetic positioning sensor 3 will not move. During use, the position changes of the sensors are shown in FIG. 3 and FIG. 4.

The real-time movement trajectory of the puncture needle body portion 2 can be obtained by acquiring a trajectory of the second electromagnetic positioning sensor 4 sliding on the puncture needle body portion 2. The real-time movement trajectory is compared with an ideal trajectory (i.e., the system-default needle insertion trajectory 6) of the electrode needle without bending. An accurate puncture position of the puncture needle body portion 2 can be adjusted by performing real-time remind for the operations.

By comparing the coordinate positions of the second electromagnetic positioning sensor 4 and the first electromagnetic positioning sensor 3, a distance value between the first electromagnetic positioning sensor 3 and the second electromagnetic positioning sensor 4 can be calculated, and then a distance between the second electromagnetic positioning sensor 4 and the tip can be obtained by calculation, thereby inferring the needle insertion depth. When the puncture needle body portion 2 just touches the skin for puncture, because the second electromagnetic positioning sensor 4 is close to the tip of the puncture needle body portion 2, the spatial position of the second electromagnetic positioning sensor 4 can be used to be close to the tip of the electrode needle, as a positioning point of puncture. The precision of this point is higher than that of the second sensor position in the actual use process.

That is, in specific implementations, the ultrasound image is first transformed into an electromagnetic positioning coordinate system (i.e., in the reference coordinate system). A plurality of electromagnetic positioning sensors on the puncture needle body portion 2 are transformed into the electromagnetic positioning coordinate system. Then, a three-dimensional ultrasound image of a scanned object in the electromagnetic positioning coordinate system is reconstructed. In the above steps, pixel points of the two-dimensional or three-dimensional ultrasound image and pixel points of an image of the puncture needle body portion 2 are unified under the electromagnetic positioning coordinate system, and a relative position relationship between the puncture needle body portion 2 and a two-dimensional ultrasound plane image and a position of the current two-dimensional ultrasound plane in the reconstructed three-dimensional ultrasound image can be calculated under this coordinate system.

Conventional intraoperative ultrasound presents a two-dimensional section with limited overall presentation of the scanned portion. With the help of the position of each frame of ultrasound image calibrated in the above processing, the three-dimensional reconstruction is carried out after interpolation calculation. After a target object is scanned, a specific three-dimensional reconstruction method based on pixels, voxels or functions may be adopted, and multi-thread or CPU parallel fast calculation may be used to realize fast (real-time) and accurate three-dimensional target reconstruction. The specific reconstruction process can be directly processed by relevant software in the prior art, and will not be described in detail in the present application.

For intraoperative ultrasound under a laparoscope, traditional segmentation algorithms may be used to segment a target area and perform the three-dimensional reconstruction in the above steps, thereby providing more accurate target reconstruction.

The above-mentioned coordinate transformation process and the calculation of a relative distance can be implemented by adopting the existing coordinate transformation software and coordinate calculation software in the prior art. In the present invention, a related algorithm is used to execute operation logics of the minimally invasive puncture system based on the navigation system, so the specific algorithm and coordinate transformation relationship are not further described in the present application.

To apply the above scheme to the ablation process, the specific processing of an ultrasound image algorithm may be as follows.

1. Fast (real-time) and accurate puncture can be achieved through multi-threaded or GPU parallel fast calculation.

2. With the help of the position of the calibrated ultrasound image, the configuration of a two-dimensional ultrasound image and three-dimensional volume data is realized, a mapping relationship between the ultrasound image and three-dimensional volume data is established, and a target detection and tracking network is used to determine relative positions of the ablation target and the ablation probe.

3. The relative spatial position of the electrode needle at the lesion, as well as the needle entry path are obtained.

As shown in FIG. 6A and FIG. 6B, an image on the left side in the figure is an original real-time ultrasound image, and an image on the right side is the generated device navigation screen, wherein the positions of the real-time needle insertion trajectory and the system-default needle insertion trajectory 6 are drawn respectively, and the operator may adjust the position of the ablation probe according to the images to realize better ablation.

The minimally invasive puncture system based on the navigation system of the present invention has the following beneficial effects.

1. Compared with a puncture device without a navigation positioning sensor, the minimally invasive puncture system based on the navigation system of the present invention is used such that the spatial position of the tip of the puncture needle body portion may be tracked and displayed in real time, so the operator can adjust a puncture direction of the puncture needle body portion according to the spatial position of the puncture needle body portion.

2. Two or more electromagnetic positioning sensors are placed on the puncture needle body portion, wherein one electromagnetic positioning sensor is designed in a fixed mode as basic coordinates of the puncture needle body portion, and the other electromagnetic positioning sensors obtain their relative positions on the puncture needle body portion through algorithms. This method not only gives designers more convenience, but also improves the precision of the system.

3. The spatial position of the puncture needle body portion in the puncture instrument is positioned and displayed through electromagnetic navigation, and the spatial position of the tip of the puncture needle body portion can be acquired before the puncture needle body portion is inserted into a solid organ. In the actual use process, the spatial position can assist the puncture needle body portion in planning a needle entry point position where the puncture needle body portion and the trachea surface are crossed.

4. The reconstructed ultrasound image is displayed on the display in real time, and in a real-time displayed image, the position of the puncture needle body portion is dynamically displayed in a display screen, which facilitates real-time adjustment of the puncture needle body portion during actual use through the displayed dynamic image. It is ensured that the puncture needle body portion is placed accurately on the target ablation lesion area.

The specific preferred embodiments of the present invention are described in detail above. It should be understood that a person of ordinary skill in the art can make many modifications and changes according to the conception of the present invention without creative labor. Therefore, all technical solutions that can be obtained by a person skilled in the art through logical analysis, reasoning or limited experiments on the basis of the prior art in accordance with the conception of the present invention shall be within the protection scope as determined by the claims.

What is claimed is:

1. A minimally invasive puncture system based on a navigation system, comprising:
a puncture instrument, the puncture instrument comprising a handheld portion and a puncture needle body portion, the puncture needle body portion being disposed on the handheld portion;
an electromagnetic navigation module, the electromagnetic navigation module comprising an electromagnetic positioning reading device, a first electromagnetic positioning sensor and a second electromagnetic positioning sensor, wherein the first electromagnetic positioning sensor is fixedly provided on a position of the puncture needle body portion adjacent to the handheld portion, the second electromagnetic positioning sensor is slidably sleeved on the puncture needle body portion, and the second electromagnetic positioning sensor is located on one side of the first electromagnetic positioning sensor away from the handheld portion; and
the electromagnetic positioning reading device is configured to acquire real-time coordinates of the first electromagnetic positioning sensor and the second electromagnetic positioning sensor, so as to obtain a real-time movement trajectory of the second electromagnetic positioning sensor relative to the first electromagnetic positioning sensor, thereby determining whether a real-time needle insertion trajectory of the puncture needle body portion entering a human body conforms to a system-default needle insertion trajectory.

2. The minimally invasive puncture system based on the navigation system according to claim 1, further comprising:
an ultrasound imaging module, the ultrasound imaging module comprising an ultrasound probe, and the ultrasound imaging module being configured to generate a real-time ultrasound image based on detection information from the ultrasound probe;
the electromagnetic navigation module further comprising a third electromagnetic positioning sensor, the third electromagnetic positioning sensor being provided on the ultrasound probe;
the electromagnetic positioning reading device being further configured to acquire real-time coordinates of the third electromagnetic positioning sensor, so as to determine a relative position relationship between the ultrasound probe and the puncture needle body portion according to the real-time coordinates of the first electromagnetic positioning sensor, the second electromagnetic positioning sensor and the third electromagnetic positioning sensor;
an image fusion module, configured to fuse the real-time needle insertion trajectory of the puncture needle body portion entering the human body with the real-time ultrasound image, and generate a device navigation screen in which a relative spatial position of the puncture needle body portion is fused into the real-time ultrasound image; and
a display module, configured to receive the device navigation screen generated by the image fusion module and display the device navigation screen.

3. The minimally invasive puncture system based on the navigation system according to claim 2, wherein the system-default needle insertion trajectory of the puncture needle body portion entering the human body is also displayed in the device navigation screen.

4. The minimally invasive puncture system based on the navigation system according to claim 2, wherein the image fusion module comprises:
an ultrasound image calibration unit, configured to construct a relative position and angle relationship between the real-time ultrasound image and the third electromagnetic positioning sensor, and determine a mapping relationship between each pixel point in the real-time ultrasound image and the third electromagnetic positioning sensor;
an ablation probe calibration unit, configured to construct a mapping relationship between the real-time coordinates of the first electromagnetic positioning sensor and the second electromagnetic positioning sensor and the real-time needle insertion trajectory of the puncture needle body portion; and
a coordinate unification unit, configured to transform mean-value coordinates of each pixel point in the real-time ultrasound image and the real-time needle insertion trajectory of the puncture needle body portion into a reference coordinate system formed by a magnetic field generator in the electromagnetic navigation module to generate the device navigation screen.

5. The minimally invasive puncture system based on the navigation system according to claim 4, wherein the image fusion module further comprises:
a three-dimensional reconstruction unit, configured to transform a two-dimensional image acquired by the ultrasound imaging module into a three-dimensional ultrasound image.

6. The minimally invasive puncture system based on the navigation system according to claim 2, wherein in the event of fusing the real-time needle insertion trajectory of the puncture needle body portion entering the human body with the real-time ultrasound image, and generating the device navigation screen in which the relative spatial position of the puncture needle body portion is fused into the real-time ultrasound image, the following operations are performed:
obtaining a real-time movement trajectory of the second electromagnetic positioning sensor relative to the first electromagnetic positioning sensor according to the acquired real-time coordinates of the first electromagnetic positioning sensor and the second electromagnetic positioning sensor, so as to acquire the real-time needle insertion trajectory of the puncture needle body portion entering the human body;
determining a mapping relationship between the reference coordinate system formed by the magnetic field generator in the electromagnetic navigation module and the real-time ultrasound image;
determining a mapping relationship between the real-time needle insertion trajectory of the puncture needle body portion entering the human body and the reference coordinate system; and
generating the device navigation screen in which the relative spatial position of the puncture needle body portion is fused into the real-time ultrasound image according to the mapping relationship between the reference coordinate system and the real-time ultrasound image and the mapping relationship between the real-time needle insertion trajectory of the puncture needle body portion entering the human body and the reference coordinate system.

7. The minimally invasive puncture system based on the navigation system according to claim 6, wherein the determining the mapping relationship between the reference coordinate system formed by the magnetic field generator in the electromagnetic navigation module and the real-time ultrasound image comprises:
performing coordinate system relationship transformation and unification on an ultrasound probe coordinate system where the third electromagnetic positioning sensor is located relative to the reference coordinate system, so as to determine the mapping relationship between the reference coordinate system and the real-time ultrasound image.

8. The minimally invasive puncture system based on the navigation system according to claim 6, wherein the determining the mapping relationship between the real-time needle insertion trajectory of the puncture needle body portion entering the human body and the reference coordinate system comprises:
performing coordinate system relationship transformation and unification on a puncture needle body coordinate system where the first electromagnetic positioning sensor and the second electromagnetic positioning sensor are located relative to the reference coordinate system, so as to determine the mapping relationship between the real-time needle insertion trajectory of the puncture needle body portion entering the human body and the reference coordinate system.

9. The minimally invasive puncture system based on the navigation system according to claim 6, wherein the generating the device navigation screen in which the relative spatial position of the puncture needle body portion is fused into the real-time ultrasound image according to the mapping relationship between the reference coordinate system and the real-time ultrasound image and the mapping relationship between the real-time needle insertion trajectory of the puncture needle body portion entering the human body and the reference coordinate system comprises:
determining a relative position relationship between the real-time ultrasound image in the reference coordinate system and the real-time needle insertion trajectory of the puncture needle body portion entering the human body according to the mapping relationship between the reference coordinate system and the real-time ultrasound image and the mapping relationship between the real-time needle insertion trajectory of the puncture needle body portion entering the human body and the reference coordinate system, and further generating the device navigation screen in which the relative spatial position of the puncture needle body portion is fused into the real-time ultrasound image.

10. The minimally invasive puncture system based on the navigation system according to claim 2, wherein the puncture instrument may be any one of a puncture set, an ablation device and a biopsy tool.
